(19) United States
(12) Patent Application Publication
(10) Pub. No.: US 2020/0232984 A1
(43) Pub. Date: Jul. 23, 2020

(54) Diagnostic Method and System for Diagnosis

(71) Applicant: BiosparQ B.V., Leiden (NL)

(72) Inventors: René Raymond Parchen, Leiden (NL); Servaas Antonie Morré, Beverwijk (NL)

(21) Appl. No.: 16/486,112

(22) PCT Filed: Mar. 1, 2018
(86) PCT No.: PCT/EP2018/055110

§ 371 Date: Aug. 14, 2019

(30) Foreign Application Priority Data
Mar. 2, 2017 (NL) 2018460
Jul. 21, 2017 (NL) 2019320

(51) Int. Cl. G01N 33/569; G01N 33/68

(52) U.S. Cl. CPC G01N 33/56927; G01N 33/6851

(57) ABSTRACT

The method for the diagnosis of a microbial infection in an organism makes use thereof that said microbial infection is at least partially present as elementary bodies in cellular material of the organism. The method comprises the steps of (1) providing a sample of cellular material from the organism; (2) processing the sample to obtain a test composition, that is enriched in elementary bodies, in so far as the sample contains any elementary bodies; and (3) Subjecting a volume of the test composition comprising at most a predetermined maximum of elementary bodies to a MALDI mass spectrometry method to identify presence of the microbial infection. The sample processing more particularly involves cell lysis and separation of elementary bodies from the lysed cell material. Subsequently, the elementary bodies (and any further material attached thereto) are contacted with a matrix material, that facilitates the MALDI mass spectrometry.

### FIELD OF THE INVENTION

The invention relates to a method for the diagnosis of a microbial infection in an organism, wherein said microbial infection is at least partially present as inclusion bodies in a cellular environment of the organism.

The invention further relates to a system for the diagnosis.

### BACKGROUND OF THE INVENTION

Microbial infections cause a threat to the health of organisms, such as mammals and more particularly human beings. A variety of diagnostic methods are available for the identification of such infections. However, such identification may be laborious. Furthermore, microbial infections may be asymptomatic, such that an infected host organism is not aware of the infection. This allows spreading of the infection. Moreover, some microbial infections are present intracellular infections.

A particular group of infections in the broad sense are at least partially in the present of inclusion bodies. Such inclusion bodies are nuclear or cytoplasmic aggregates of stainable substances, usually proteins. The inclusion bodies typically represent sites of viral or bacterial multiplication. In various cases, particularly within the Chlamydia and Chlamydophila genera, the inclusion bodies may contain the infectious material in two forms: one form that is suitable for multiplication and another form that is suitable for infection of cells. The latter form may leave the infected cell and spread to enter other cells. The one form is also known as a reticulate body and the other form is known as an elementary body. The two forms differ in several aspects, such as the RNA/DNA ratio, the density of nuclear material and the type of cell wall. The elementary bodies that can survive in extracellular form have a low RNA/DNA ratio, for instance below 1, have electron-dense nuclear material and a relatively rigid cell wall. They further induce phagocytosis and inhibit phagosome function. The reticulate body are thus non-infectious are metabolically active, with a higher RNA/DNA ratio, for instance 3-4. They further have a loosely distributed nucleoid, a thinner more flexible cell wall and replace by binary fission. The present invention makes use of the properties of the elementary bodies. While such elementary bodies are known to exist in Chlamydia and Chlamydophila genera, it is not excluded that other microbial infections, for instance virus or gram negative bacteria, spread through a similar mechanism of elementary bodies, i.e. having a form or phase that contains dense nuclear material and is overall relatively rigid so as to enable extracellular existence.

The presence of inclusion bodies may be due to bacteria that have infected a host organism and more particularly are intracellular bacteria. However, inclusion bodies may also result due to expression of a recombinant gene from one type of organism (such as a eukaryote cell) into another organism, such as a prokaryote cell. Examples of classes of inclusion bodies include viral inclusion bodies, inclusion bodies in red blood cells, inclusion bodies in skin conditions, Councilman bodies and Dohle bodies. Viral inclusion bodies may be intracytoplasmic, intranuclear, for instance acidophlic or basophilic and furthermore both intranuclear and intracytoplasmic. Inclusion bodies in Red Blood Cells may be as developmental organelles, protozoal inclusions and as abnormal haemoglobin precipitates. A number of important virus and infections are understood to be in the form of inclusion bodies, including negri bodies in Rabies, Cowdry type A in Herpes simplex virus and Varicella zoster virus, Tones bodies in Yellow fever and Cowdry type B in Polio and adenovirus. Examples of viral inclusion bodies in plants include aggregations of virus particles (like those for Cucumber mosaic virus) and aggregations of viral proteins (like the cylindrical inclusions of potyviruses).

A relevant bacterial infection of an intracellular type of bacteria and being at least partially in the form of inclusion bodies and comprising both reticulate bodies and elementary bodiesis found in the Chlamydia genus, and more particularly Chlamydia trachomatis. Also Mycoplasma species infections are understood to be intracellular bacterial infections. In the following, the focus will be on inclusion bodies of bacterial species of the Chlamydia genus.

Chlamydia trachomatis (hereinafter also abbreviated to CT) is the single most important infectious agent associated with blindness (trachoma); about 84 million worldwide suffer from C. Trachomatis eye infections and 8 million are blind as a result of the infection. Infection with Chlamydia trachomatis is furthermore now the most common bacterial sexually transmitted disease, affecting about 4.2% of women and 2.7% of men in the general population and up to 15% in STD clinics and over 20% in high risk groups. Both the US Centers for Disease Control and Prevention and the European Centre for Disease Prevention and Control reported an increase in chlamydia infection rate in recent years. Several factors may account for the increase of diagnosed CT infections, including changes in sexual behaviour and lack of prevention and education, but also more frequent testing with improved detection systems. Yet, many infections are asymptomatic and still remain undetected. It is estimated that 85% of women and up to 60% of men are asymptomatic resulting in many untreated CT infections. The infection is a common cause of urethritis and cervicitis but the main problem are the late complications in women including PID, Ectopic Pregnancy and Tubal Factor Infertility. These late complications are the reason for the many screening initiative to get the CT prevalence down to reduce these late complications both for the women affected as for the costs for society.

Chlamydia species are obligate intracellular bacteria, and are represented in two developmental forms in the host: (1) intracellular non-infectious reticulate Bodies (RBs) representing the bacteria in replicative form, and (2) extracellular Elementary Bodies (EBs) acting as infectious particles that target host cells. For sake of simplicity, the abbreviations RB and EB will also be used hereinafter.

EBs are able to internalize in the host cell after interactions between the bacterial outer membrane proteins and different host cell receptors. After internalization, EBs express bacterial inclusion proteins that prevent fusion with lysosomes, and transport to the microtubule-organizing center. At this location, the EBs differentiate into RBs, which are able to replicate by binary fission and subsequently re-differentiate into EBs at the end of the developmental cycle. EBs can exit the cell after lysis of the host cell or via extrusion and may to infect other host cells. Chlamydia EBs are recognized by the host organism by receptors of the innate immune systems, called pathogen/pattern recognition receptors, and induce both innate and adaptive immune reactions. However, the inflammation associated with the immune response is usually less pronounced, and most infections remain asymptomatic.

Unfortunately, current diagnosis of Chlamydia trachomatis is time consuming and expensive and no good Rapid Diagnostic Test (RDT) or so-called Point of Care (PoC) tests exist yet with high sensitivity and specificity. There two primary methods available: the old Golden Standard culture-based diagnosis for truly viable CT participles (EBs) and Nucleic Acids Amplification Tests (NAAT), which is currently the gold standard.

Culture-based diagnostic tests were long considered the reference test for CT detection. Swabs from different anatomical sites can be used as specimen for culture-based diagnosis. The sample is centrifuged down on cell monolayer of a specialized cell-line, and after 48-72 hour the cells can be analysed for the presence of characteristic intra-cytoplasmatic inclusion bodies by a highly specific staining. However, since culturing requires viable organisms, the sensitivity may be impaired by inadequate specimen collection, storage and transport, toxic substances in clinical specimens and low bacterial loads can not be detected. In addition, this method is very time-consuming and labour intensive, and is therefore rarely used nowadays in diagnostic laboratories except in child abuse cases. Moreover, its sensitivity as compared to NAATs is in the best culture labs max 50-60%. NAATs are currently generally considered as the gold standard for CT diagnosis. NAATs do not depend on viable pathogens and higher loads, and are therefore much more sensitive than culture.

Most of the NAATs are based on polymerase chain reaction (PCR) and use fluorescence labelled probes to detect the amplified PCR products in real time. Nowadays, NAATs often target two regions, since problems regarding deletions and recombinant of the target regions including even plasmid free variant have been shown in the past and the plasmid region is the preferred region since it is 10× more present as compare to chromosomal targets. In principle, all relevant clinical materials can be analysed by NAATs, however first void urine is the recommended sample type in men, and vaginal swabs are the recommended sample type for women. Women are more often tested as compared to men based on the late complication burden women have in contract to men. NAATs and culture-based test are usually performed in a central laboratory and require transportation of specimens and transmission of test results to the clinicians. As a consequence, they also require a second visit of the patient. Delayed treatment, or no treatment at all if patients do not re-appear again (especially low social, economic and educational settings), could contribute to the high incidence of infection. It is therefore desired to develop a Rapid diagnostics tests (RDTs) that could be performed near-patient (Point-of-Care). Such RDT would further allow immediate antibiotic treatment in case of a positive test and no loss of society on an economical level by a second visit. However, compared to culture and NAATs these current RDTs are significantly less sensitive and specific and not implemented anywhere. RDT development is top priority to combat CT infections in both developed and underdeveloped countries.

Furthermore, it is known to apply MALDI-TOF MS (i.e. Matrix-Assisted Laser

Desorption/Ionization Time-of-Flight Mass Spectrometry) to detect an infection of pathogens present in a bodily fluid. This technique is specified in WO2009/065580 and further discussed in a conference paper presented at the 2008 Annual Meeting of the Infectious Diseases Society of America by the inventor and co-authors C. Boogen, M. Schwarz and M. Kostrzewa. As presented in the abstract, urine (1 ml) was centrifuged in two steps (30 sec at 2000 g and 5 min at 15,500 g) to obtain pellets. Proteins were then extracted from pellets by adding formic acid and acetonitrile. Several bacteria could be correctly identified by means of this method. According to WO2009/065580, essentially the same method could further be used to identify viruses, such as Rickettsia and Chlamydia. Thereto, however, ultracentrifugation is needed to obtain the virus in the form of virions in sufficiently large quantities. The term ‘virion’ is known to refer to the virus particle as opposed to the infected cell. The virus particle contains a capsid as an outer protein shell. In the method presented in WO2009/065580, the virus particle is decomposed according to a special decomposition process so as to obtain the coat protein of the capsid and dissolve them for incorporation into the matrix crystals. It appears that the disclosed method is a first step for concentrating the virus particle in a bodily fluid to pellets, and a second step for decomposition of the pellets of virus particles, after which the relevant protein will be present in a supernatant.

The description is however problematic in more than one aspect. First, No information is presented on said special decomposition process. Furthermore, no identification is given what would be sufficiently large quantities. Such anyhow appears difficult for an effective and timely detection of Chlamydia. In fact, it would be desired to obtain a method that allows detection of Chlamydia before the virus has developed into a disease into the patient, in other words, when the effective quantity of the virus is still low.

Therefore, there is still a need for a proven method so as to enable the identification of Chlamydia by means of MALDI mass spectrometry.

### SUMMARY OF THE INVENTION

It is therefore an object of the present invention to provide an improved method for the diagnosis of a microbial infection in an organism, such as the diagnosis of Chlamydia trachomatis, which has a clinical sensitivity that is at least comparable to that of NAAT-type tests and which does not require a second visit of a patient to a clinician.

It is furthermore an object of the present invention to provide a system for the diagnosis of the microbial infection, specifically Chlamydia trachomatis.

According to a first aspect, the invention provides a method for the diagnosis of a microbial infection in an organism, wherein said microbial infection is at least partially present as elementary bodies in cellular material of the organism, which method comprises the steps of:
Providing a sample of cellular material from the organism;
Processing the sample to obtain a test composition, that is enriched in elementary bodies, in so far as the sample contains any elementary bodies, and
Subjecting a volume of the test composition comprising at most a predetermined maximum number of elementary bodies to a MALDI mass spectrometry method to identify presence of the microbial infection.

According to a second aspect, the invention provides a system for diagnosis of a microbial infection, which is at least partially present as elementary bodies in cellular material of an organism, comprising in combination (1) means for lysis of a sample of cellular material; (2) separation means for separating of elementary bodies from other material obtained by cell lysis; (3) mixing means for mixing a composition of a matrix material with the thus separated elementary bodies to obtain a test composition comprising said elementary bodies, (4) a dispensing unit for dispensing a volume of said test composition comprising a predetermined maximum of elementary bodies (5) a MALDI mass spectrometer configured for generating a mass spectrum of said test composition, and (6) a processor for generating a mean spectrum from a plurality of mass spectra for individual volumes and for comparing the mean spectrum with a reference, typically from a database.

According to a third aspect, the invention comprises a system for identification of elementary bodies, comprising in combination (1) means for lysis of a sample of cellular material; (2) separation means for separating of elementary bodies from other material obtained by cell lysis; (3) mixing means for mixing a composition of a matrix material with the thus separated elementary bodies to obtain a test composition comprising said elementary bodies, (4) a dispensing unit for dispensing a volume of said test composition comprising a predetermined maximum of elementary bodies (5) a MALDI mass spectrometer configured for generating a mass spectrum of said test composition, and (6) a processor for generating a mean spectrum from a plurality of mass spectra for individual volumes.

In again a further aspect, the invention relates to the use of the systems of the invention for identification of elementary bodies and/or for diagnosis of a microbial infection. The use of the identification of elementary bodies in an organism, comprises: (1) providing a sample of cellular material from the organism; (2) processing the sample to obtain a test composition, that is enriched in elementary bodies; (3) subjecting at least part of the test composition to a MALDI mass spectrometry method to identify said one or more elementary bodies.

The invention is based on the insight, that elementary bodies can be effectively separated from other cellular material so as to obtain a test composition, wherein the proteins in the elementary body are detectable by means of mass spectrometry. More particularly, it is feasible to generate volumes of the test composition comprising a limited number of elementary bodies per volume. The limited number is for instance at most 10, preferably at most 5 and more preferably at most two. One elementary body per volume is most preferred. This allows using mass spectrometry with a high signal to noise ratio. Furthermore, it is feasible to subject a series of volumes to mass spectrometry sequentially. Since each volume has substantially the same content (i.e. a limited and likely predefined number of elementary bodies), it is easy to combine and/or analyse resulting spectra and/or other results in a meaningful manner.

Furthermore, the concentration of proteins in the elementary bodies is beneficial for diagnosis by means of a MALDI mass spectrometry method. A particularly preferred type of MALDI mass spectrometry is known as MALDI-TOF MS, which is abbreviation for matrix-assisted laser desorption/ionization time-of-flight mass spectrometry. This is an emerging technique for high-throughput, cost-effective and rapid microbial identification. Herein, microbes are identified by comparing their protein fingerprint (i.e. their total protein content) to a reference, typically from a library.

In a preferred embodiment, the method further comprises the step of generating droplets of the test composition, which droplets are the volumes sequentially subjected to the MALDI mass spectrometry method. The droplets are typically generated from a microfluidic dispenser chip on the basis of actuation carried out by a known actuator, such as for instance a piezoelectric actuator. The volume of the droplets is suitably in the range of picoliters, for instance 1-10 picoliters.

In a highly relevant implementation hereof, a selection step is carried out after generation of the droplets. Such a selection step ensures that each volume has less elementary bodies than the predefined maximum. More particularly, the step is carried out so as to ensure that each volume has contains at least a minimum number of elementary bodies. Preferably, all volumes contain the same number of elementary bodies, and more preferably, this number is one. However, it is not excluded that the number of elementary bodies is greater than one, at least in part of the volumes. Furthermore, it is feasible that volumes without elementary bodies are also generated. Such volumes can be used to identify a background. In a further processing step, the spectrum of such volume without elementary bodies can be subtracted from the spectrum of a volume with one or more elementary bodies. This is deemed to further increase the signal-to-noise ratio.

Preferably, the selection step is carried out by optical image analysis. The optical image analysis is for instance embodied with a camera. Droplets or other volumes that are not selected for measurement by mass spectrometry may be removed from the droplet stream, for instance by means of an electromagnetic shutter. Preferably, the droplets are viewed at a location adjacent to an orifice of the droplet dispenser. The viewed droplet is then to be ejected from the orifice as a free flying droplet upon a next dispensing event. In one version, the particles are irradiated with a suitable wavelength, so as to effect fluorescence of specific substances in the particles, more particularly aerosol particles. A suitable irradiation source is an excitation laser. Alternatively, these droplets are selectively not ionized, i.e. not treated with a laser for ionisation.

One advantage of optical image analysis is that the optical image of a specific volume may be registered. The registration may be either in the form of an image, or optionally in a form of analysis data. Such data could for instance be an image showing the contrast, and/or an indication of the number of dots in the image corresponding to the number of elementary bodies. The storage in the form of analysis data may be suitable so as to limit the amount of data to be stored. The stored image or the analysis data thereof may be used by a processor, so as to generate a sample results based on a plurality of spectra generated by the mass spectrometer. This is feasible, as the image or analysis data thereof can be directly linked to one individual generated spectrum. Furthermore, in the event that the subsequent optical images demonstrate absence of any elementary bodies, the processor may enter into a specific protocol. Such negative result in the optical imaging can either mean that there are no elementary bodies present, or that an error has occurred. One part of the protocol is then an algorithm to verify an appropriate set up of the processing and the dispensing. In some embodiments, this may be implemented as the provision of a signal or alarm to an operator. According to another implementation, a predefined number of droplets apparently devoid of elementary bodies may nevertheless be selected. The resulting mass spectrometry is then used for verification of the apparent negative result.

Preferably, the processing of the sample into a test composition comprises the steps of cell lysis and of separation of the elementary bodies from other parts of the lysed cell material. It has been understood by the inventors that cell lysis and subsequent separation allows maintaining the integrity of the elementary bodies, while other cellular material, including for instance reticulate bodies and cell membranes is disintegrated into cell debris and then removed. While several methods are known for cell lysis, a most preferred method is sonication. This method is capable of lysing the cell and destroying various cell parts, while leaving the more rigid elementary bodies intact. Another lysing technique such as liquid homogenization, mechanical, electric (electroporation), chemical, possibly in combination with bead beating, is however not excluded. In sonication, high frequency sound waves shear cells. The power used for sonication is for instance in the range of 40-100 W, for instance 50-80 W. Preferably, it is performed using an ultrasonic bath or an ultrasonic probe. The power source attached to the probe generates sound energy electronically, for instance within a range of 20-50 kHz. Preferably, the sonication is carried out in a plurality of periods with intermittent periods. Suitably, vortexing is carried out in between said sonication periods. The sonication periods are suitably in the range of 15-40 seconds, such as 20-30 seconds, the intermittent periods may be as short as 0.5-5 seconds. Variations hereof are not excluded. These sonication settings are for conventional cell lysis. It was found that such settings result therein that the cell wall is lysed, but the elementary bodies are not lysed.

In a further implementation, the separation step is carried out by a centrifuge. This centrifuge treatment is configured so that the elementary bodies end up in the supernatant and separated from the cell debris. Most suitably, the composition with the elementary bodies is thereafter concentrated. Here again, a centrifuge treatment may be applied, wherein the elementary bodies would be pelletized. Alternatively, use can be made of another separation technique, such as filtration and immunomagnetic separation. This further concentration of the elementary bodies is understood to increase the resolution of the subsequent measurement by means of MALDI mass spectrometry. For sake of clarity, it is observed—in comparison to the cell lysis technique for bacteria disclosed in WO2009/065580A1 that the present invention carries out a centrifuge treatment after the cell lysis, to remove cell debris from the elementary bodies. The separated elementary bodies were processed to obtain pellets.

Preferably after this concentration step, a composition comprising a matrix material is added to obtain a test composition. The use of a matrix material is necessary in MALDI methods, as apparent from the full name of MALDI: Matrix assisted laser desorption ionisation. Preferred matrix materials are known from EP2210110B1, which is herein included by reference. The matrix material is typically provided as a composition in a volatile solvent and furthermore one or more additives. An alcohol, such as ethanol, is a preferred solvent. The additives are for instance present for control of the pH of the test composition. The additives for instance include water and a volatile acid, such as trifluoroacetic acid. The term ‘volatile’ is used in the context of the present invention to refer to a compound that can be evaporated at a temperature ranging from room temperature to 100° C. More preferably, use is made of a type of MALDI TOF MS, known as single cell MALDI TOF MS. This is a technique, known per se, wherein the sample preparation is such that each sample detected by the mass spectrometer contains material from a single cell.

In one implementation, the mixing with the composition of the matrix material for use in the MALDI mass spectrometry method is carried out by resuspension of the pelletized elementary bodies. It is observed that in one embodiment, the enrichment of the test composition involves a substantial isolation of the elementary bodies. However, this is not deemed necessary. It may also be that the test composition is enriched with elementary bodies, while other solid matters remain part of the composition. Suitably, the amount of the elementary bodies is at least 50% by weight based on total solid matter in the test composition. More preferably, the amount is at least 70% by weight based on total solids, or even at least 80%.

Subsequent to the dispensing of droplets and any optional selection of droplets the matrix material will be crystallized onto the solid matter, more particularly the elementary bodies. Herein, a volatile solvent of the composition of the matrix material is evaporated. Herein, a stream of coated droplets is generated that is also known as an aerosol beam. The analyte with the crystallised material on a surface of the one or more elementary bodies is then irradiated with a laser, typically using UV light. This has the effect that the crystallized matrix absorbs the energy of the laser light and transmits it to the elementary bodies; direct irradiation of the elementary body would rather destruct such bodies. Furthermore, charged particles, such as protons, will be generated due to the absorption of the laser light, which result in ionisation of the proteins. This ionisation of the proteins forms the basis for a successful time-of-flight mass spectrometry measurement. In one further implementation, the droplets are furthermore transmitted from ambient atmosphere into a vacuum atmosphere. The vacuum atmosphere is herein an atmosphere at a pressure sufficiently low to allow mass spectrometry.

It is a first advantage of the method of the invention, that the sample collection is not considered to be critical. In case of diagnosing CT, a sample may be collected by a patient undergoing the diagnosis or may be collected by a clinician. Based on preliminary experience, all samples containing bacteria cells in suspension are appropriate samples for single-particle MALDI-TOF MS analysis, including first void urine and (the stabilization buffer of) swabs.

It is a further advantage of the method of the invention, that the diagnosis can be performed in a short period. Preliminary experiments indicate a duration of 15-30 minutes, such as 20 minutes, whereas the NAATs test easily takes 5 hours. This allows application of the method by means of a point-of-care treatment, i.e. wherein a patient can wait for the outcome of the diagnosis. If the diagnosis is positive, the patient may be prescribed a treatment in the form of a medication, such as antibiotics.

Beyond that, it appears that the overall resolution of this method is good. The large density of proteins in the elementary bodies facilitates an appropriate detection by MALDI MS, which is dependent on proteins. The definition of droplets each containing a predefined limited number of elementary bodies is furthermore helpful as it allows combination of spectra based on individual droplets in a meaningful manner For instance, a mean spectrum is generated on the basis of a plurality of spectra of individual droplets. The number of spectra is suitably 10-200, for instance 20-100 or 40-80 spectra. This ability to obtain a spectrum with adequate signal-to-noise ratio therewith not merely allows to confirm that there are elementary bodies indicating an infection, but also to identify the type of infection, and more particularly the specific microorganism responsible for the infection.

The method has been tested and has been found feasible to detect Chlamydia in urine, in an anal swab and a vaginal swab, wherein the pathogen concentration was equal or less than 10⁴ CFU/ml, for instance 10³ CFU/ml, as typical for a swap. The present invention has the potential to detect Chlamydia on the basis of the presence of at little as 10 CFU/ml.

It will be understood that while the present method is primarily intended for the detection of a microbial infection, such as an infection with CT, it is not excluded that the method is applied for identification of elementary bodies as such.

### BRIEF INTRODUCTION OF THE FIGURES

These and other aspects of the invention will be further elucidated with reference to the figures, which are purely diagrammatical and not drawn to scale, wherein:

FIG. 1 shows a schematic representation of an apparatus for MALDI mass spectrometry with a preferred pre-treatment for a liquid test composition, and

FIG. 2 shows a schematic representation of the particle flow path and mass spectrometer within the apparatus of FIG. 1.

FIG. 3A-B diagrammatically indicates process steps according to one embodiment of the method of the invention, wherein FIG. 3A indicates the generation and selection of droplets and FIG. 3B indicates MALDI TOF mass spectrometry.

FIG. 4 shows a flow chart for the processing of the sample according to one embodiment of the method of the invention;

FIG. 5 shows a mass spectrum obtained in a preliminary experiment according to the invention

### DETAILED DESCRIPTION OF ILLUSTRATED EMBODIMENTS

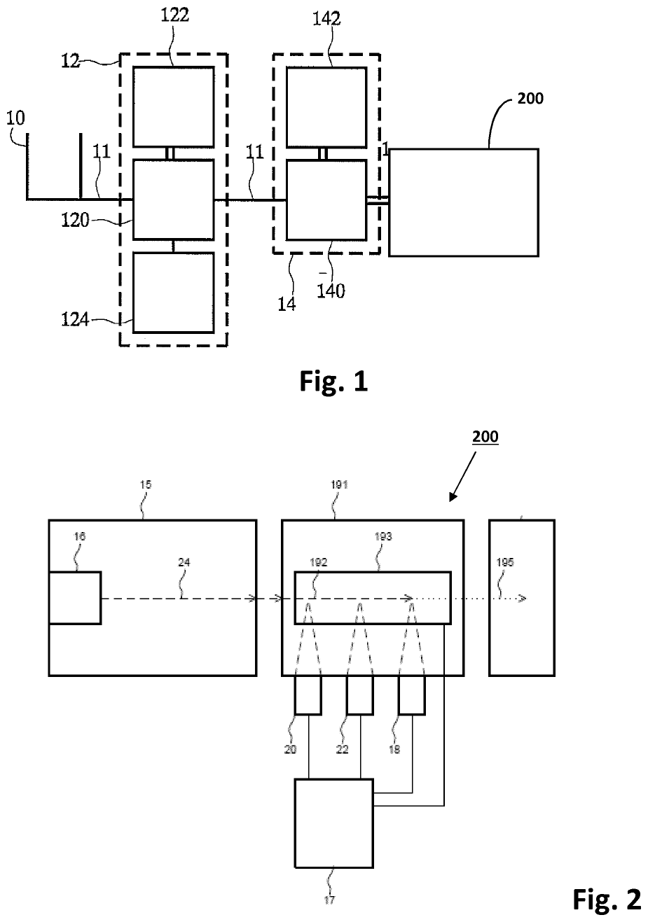

FIG. 1 shows a schematic representation of a first embodiment of an apparatus for MALDI mass spectrometry. FIG. 2 shows in more detail the portion 200 of the apparatus, hereinafter also referred to as a flight path unit 200. MALDI mass spectrometry is particularly suitable for identification of biological material. One preferred type of biological material is micro-organisms such as bacteria, fungi and viruses. Other types of biological material that can be identified with MALDI include for instance blood cells, peptides.

The apparatus comprises a sample receiver 10, conduits 11, a first mixing unit 12, a second mixing unit 14, and a flight path unit 200. The flight path unit comprises a drying chamber 15, an ionization chamber 191 and a time-of-flight tube 194. A droplet is ejected by any droplet ejector 16, such as for instance based on a piezoelectric resonator. The droplet follows a droplet beam 24 that extends from the drying chamber 15 into the time-of-flight tube 194. Upon drying the droplet beam 24 is actually converted into a particle beam 192. Upon ionization by radiation from a pulse laser 18, the particle beam 192 is converted into a ion beam 195. The mass spectrometer—not shown—measures the ions of the ion beam 195 and creates spectra on the basis thereof According to one embodiment of the invention, use is made of a sensor 20, 22 for determining a morphology parameter so as to select particles that are ionized by a laser pulse of the pulse laser 18. This is particularly done so as to ionize only those particles that may lead to useful spectrum information.

The first mixing unit 12 comprises a first mixer 120, a container 122 for solvent and/or antisolvent, such as water, and a detector 124. Rather than one container 122, two separate containers may be present. Sample material that is for instance obtained from a patient, is diluted with the solvent and/or antisolvent in the first mixer 120. Detector 124 is suitably an optical detector configured to detect light scattered from individual micro-organisms when the micro-organisms flow through a measurement beam. From a count of micro-organisms that are detected on average per unit of time interval, the density may be determined. Such detector 124 is known per se and is for instance a cytometer or flow cytometer. Particle detector 124 is shown coupled to a control input of first mixer 120. The control mechanism is arranged to increase the amount of solvent and/or antisolvent, until the measured density has dropped to or below a predefined density. Preferably both are added in a predefined ratio. A liquid circulation circuit may be used to circulate the composition until the desired density has been achieved. The second mixing unit 14 comprises a second mixer 14 and a matrix material reservoir 142. Matrix material reservoir 142 is coupled to the second mixer 140. The second mixer 14 is configured to mix the matrix material into the test composition obtained from the first mixing unit 12.

The droplet generator 16 may be provided with means for evaluation whether a droplet contains a single microorganism or any other number of microorganisms. Such a detecting means may be arranged to view the suspension in a channel prior to ejection by a nozzle. The generator 16 may further be provided with means for directing an ejected droplet to a first position or to a second position depending on information obtained from the detecting means. The first position is then a target position, i.e. a flow path towards the position where a laser source may eject radiation on the particle so as to ionize it. The second position is a waste position. The directing means are configured for deflection of the droplet or a motorized stage configured for directing the nozzle. Such an apparatus is known per se from EP2577254B1, and is included herein by reference.

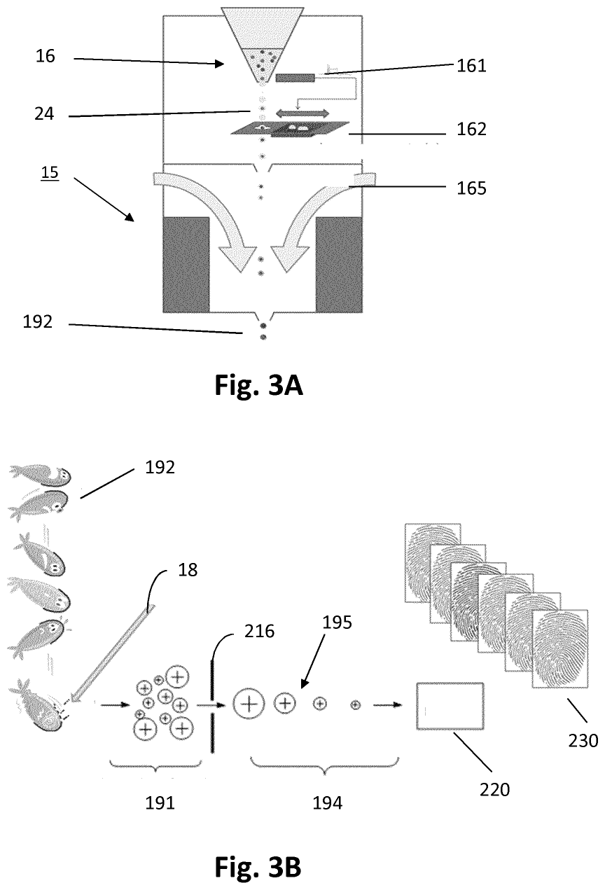

FIG. 3A shows one embodiment of a droplet generator 16 and the chamber 15 in more detail. In this figure, the flow path of the droplet beam 24 through the chamber 15 may have a vertical orientation. In this embodiment, the detecting means 161 are arranged to optically sense the droplets upon ejection from the nozzle of the droplet generator 16. The sensor 161 is coupled to a selection means 162, preferably an electromagnetic shutter, to remove those droplets that do not contain any microorganism.

Due to the small droplet size, it has been found that the droplets quickly, i.e. in the first few centimeters of the flow path, arrive at a constant velocity. This velocity is a balance of gravity and aerodynamic resistance. Due to the drying, the droplet beam 24 is converted into particle beam 192. The chamber 15 is provided with temperature controlled walls so as to keep the temperature in the chamber 15 constant. In one embodiment a temperature of 22-30° C. is chosen. The chamber 15 is further provided with an inlet 165 for gas generating a homogeneously distributed sheath flow. The gas comprises for instance air or nitrogen and is controlled with respect to the concentration of water vapor and optionally any solvent. Suitably, the water vapor concentration is controlled, for instance such that the relative humidity is 30% or more. During flight through the drying chamber 15, the matrix material in a liquid drop crystallizes on the analyte, typically a microorganism, while the drop dries in flight, resulting in a dried particle, which is also referred to as the test sample. Typically, the drop is launched with a diameter in the range of 30-60 μm. The dried particle has an aerodynamic diameter of less than 3.0 μm in a first embodiment, wherein the test sample contains a single bacteria. The sheath flow transports the droplets towards the inlet of the aerosol time-of-flight mass spectrometer.

FIG. 3B illustrates the identification process based on the generated test samples in a particle beam. A laser pulse is fired at the dried particle from pulse laser 18. This results in ionization of material from the test sample. The ionized material is then accelerated in a ionization chamber 191, in which high voltages are present to accelerate the ionized material. The ionized elements passes a charged grid 216. As a consequence, individual ions of a ion beam 195 are separated in a drift region 194, that is free of an electric field. This drift region is also known as a time-of-flight tube. The separated ions are detected by a detector 220. The processor that is coupled thereto processes the obtained data to generate a spectrum or data set 230 (‘fingerprint’) that can be compared with known data sets. Such known data sets are typically stored in a library.

### EXAMPLE

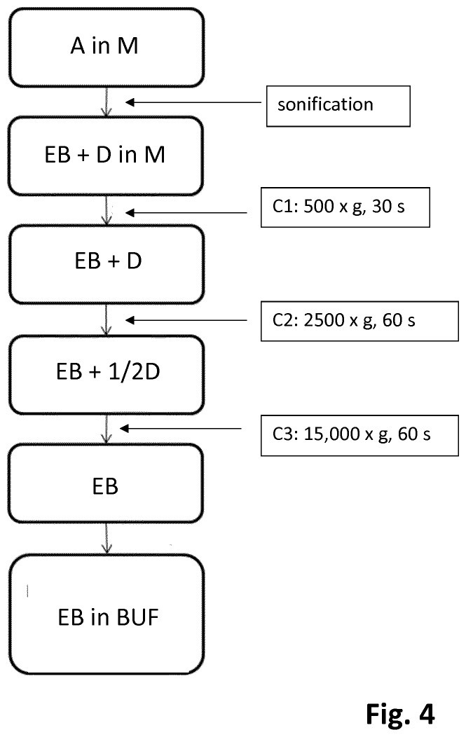

A test was carried out with Chlamydia trachomatis species that had been grown in a cell line of HeLa cells. The latter is a cell type in an immortal cell line used in scientific research. The sample preparation is shown in FIG. 4. The starting composition contained A (the said species in the HeLa cells) in a medium M. The HeLa cells were lysed by sonication, using three sonication periods each of 20 seconds at 70 W, interrupted by intermittent periods of 2 seconds using vortexing. This resulted therein that the elementary bodies (EB) could be collected, while other cell material was destructed to cell debris (D). After the sonication there was thus EB+D in medium (M). The cell material was then transferred to a centrifuge, for separation of the cell debris (D) and the elementary bodies (EB). Three subsequent steps (C1, C2, C3) were carried out in this example, using increasing spinning rates, i.e. 500× g, 2500× g and 15,000× g. As a consequence, first the medium M was removed, then a portion of the cell debris D was removed, and then the remaining cell debris D was removed. This led to pellets of elementary bodies.(EB). In the removal of the cell debris, typically first the more rough cell debris is removed and thereafter the finer cell debris. The figure refers for sake of simplicity to 1/2D, as if both steps each remove 50%. That is merely schematically and not a hard requirement. It will be understood that the exact spinning rates can be amended Furthermore, while in one embodiment according to the invention, three steps are carried out, this number may be varied. The pellets were then resuspended into a buffer (BUF) for storage (for instance at −80° C.), or for direct use.

While the figure shows a specific embodiment for characterization of the method of the invention, it will be understood that when used for diagnostic purposes, the use of C. trachomatis and HeLa cells is typically replaced by the use of cells that could contain the C. trachomatis. In such diagnostic method, the cells may be mixed with a medium, before the destruction step. Following the destruction step, the elementary bodies EB will be separated from the cell debris and the medium. The use of a centrifuge is therein preferred, although the centrifuge may be replaced in part or entirely by other separation techniques, including filtering, membrane filtering and/or activated (para)magnetic beads.

A test composition was then generated by bringing the pellets of elementary bodies (EB) into contact with a matrix material. In this example, use was made of resuspension, with 2-mercapto-4,5-dimethylthiazole as the matrix material. Droplets were generated with a droplet generator. Each droplet has a volume in the range of 10-100 picoliter. An image sensor, more particularly a camera, was provided at a location so as to enable recording of the droplets at the exit of the droplet dispenser. The image sensor was coupled to a processor to check whether dot-shaped elements, typically darker than other parts of the droplet were present in the droplet. A droplet was selected when the droplet contained one dot-shaped element. If a droplet was not selected, it was taken out of a droplet beam by means of an electro-magnetic shutter. The droplets thereafter passed a drying section. This resulted in the generation of coated particles suitable for MALDI mass spectrometry.

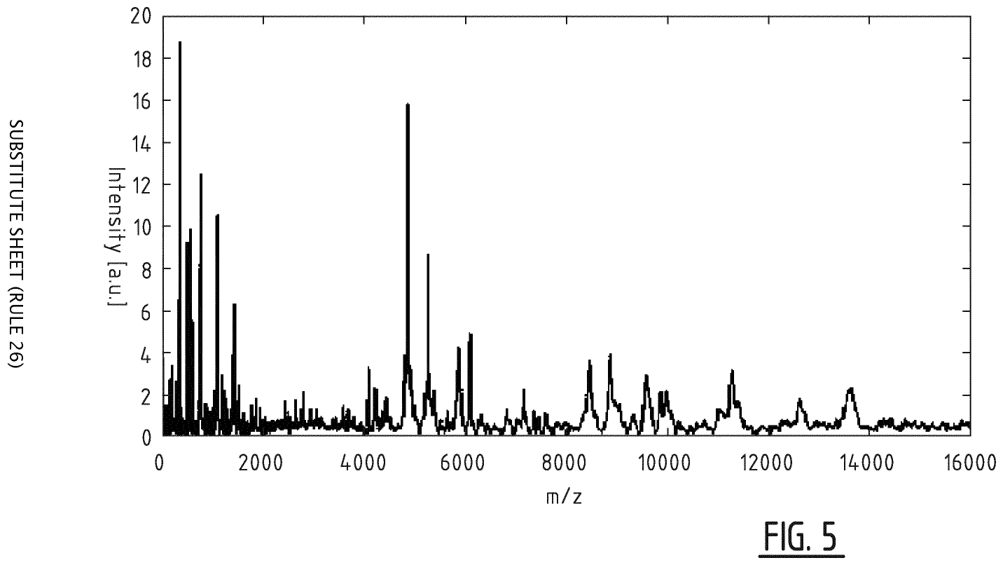

MALDI mass spectrometry on the individual droplets was thereafter carried out. Mass spectra of individual droplets were generated. The spectra contained a sufficient signal were accumulated, such that signal-to-noise ratio became sufficiently large to distinguish 20-30 significant peaks within the spectrum. Herein, the base line, which corresponds with the spectral level caused by a signal part that varies from particle to particle, is subtracted from the signal. The intensity has been normalized with local variance of the height of the base line. A peak with an intensity of at least one is considered sufficiently significant. The spectrum shown in FIG. 5 was obtained.

In case that it is expected that other objects than only elementary bodies are present, a classification may be carried. Use can be made therein of the method specified in EP2836958, which is included herein by reference.

1.-24. (canceled)

25. A method for the diagnosis of a microbial infection in an organism, wherein said microbial infection is of a type that is present in one form suitable for multiplication known as a reticulate body and another form suitable for infection of cells known as elementary body, wherein said microbial infection is at least partially present as elementary bodies in cellular material of the organism, which method comprises the steps of:
providing a sample of cellular material from the organism;
processing the sample comprising the steps of lysing the cellular material so as to maintain the integrity of the elementary bodies, and separating the elementary bodies from the lysed cellular material into an enriched sample, and adding a composition of a matrix material thereto, therewith generating a test composition, that is enriched in elementary bodies, in so far as the sample contains any elementary bodies; and
subjecting a volume of the test composition comprising at least one and at most a predetermined maximum of elementary bodies, in so far as the sample contains any elementary bodies, to a MALDI mass spectrometry method to identify presence of the microbial infection.

26. The method as claimed in claim 25, wherein the test composition that is enriched in elementary bodies comprises at least 50% by weight of elementary bodies, based on total solid matter in the test composition.

27. The method as claimed in claim 25, wherein the method further comprises the step of generating droplets of the test composition, which droplets are the volumes sequentially subjected to the MALDI mass spectrometry method.

28. The method as claimed in claim 27, wherein the step of subjecting a volume of the test composition to a MALDI mass spectrometry method comprises, subsequent to dispensing of said droplets, the steps of:
crystallizing the matrix material onto solid matter, more particularly the elementary bodies, wherein a volatile solvent of the composition of the matrix material is evaporated, therewith generating an aerosol beam of elementary bodies coated with matrix material;
irradiating said coated elementary bodies with a laser, resulting in ionization of proteins; and
performing a time-of-flight mass spectrometry measurement on said ionized proteins.

29. The method as claimed in claim 25, wherein the predetermined maximum of elementary bodies per volume is 10.

30. The method as claimed in claim 29, wherein each volume contains at most 2 elementary bodies.

31. The method as claimed in claim 27, wherein the generated droplets are subjected to a selection step, which comprises registration of an optical image of a droplet, and selection of droplets based on analysis of the optical image.

32. The method as claimed in claim 25, wherein the lysing is carried out by means of sonication.

33. The method as claimed in claim 25, wherein the separation involves a centrifuge treatment.

34. The method as claimed in claim 33, wherein the centrifuge treatment provides a supernatant comprising the elementary bodies, separated from cell debris.

35. The method as claimed in claim 25, further comprising a step of concentrating a composition of the separated elementary bodies.

36. The method as claimed in claim 25, wherein the microbial infection is generated by at least one intracellular bacterial species.

37. The method as claimed in claim 36, wherein the bacterial species belongs to the Chlamydia genus.

38. The method as claimed in claim 25, wherein the microbial infection is asymptomatic.

39. The method as claimed in claim 25, wherein said MALDI mass spectrometry method comprises:
processing the volume of the test composition, preferably the droplet, to crystallize the matrix material thereof onto the at least one elementary body contained in the volume, therewith obtaining an analyte;
ionizing at least part of the analyte;
separating the ionized components using a time-of-flight detector to obtain a spectrum; and
comparing the spectrum with at least one reference for identification of the microbial infection.

40. The method as claimed in claim 25, wherein the organism is a mammal.

41. A system for diagnosis of a microbial infection of a type that is present in one form suitable for multiplication known as a reticulate body and another form suitable for infection of cells known as elementary body, wherein said microbial infection is at least partially present as elementary bodies in cellular material of the organism, said system for diagnosis comprising in combination:
means for lysis of a sample of cellular material configured to maintain the integrity of the elementary bodies;
separation means for separating of elementary bodies from other material obtained by said means of cell lysis;
mixing means for mixing a composition of a matrix material with the thus separated elementary bodies to obtain a test composition comprising said elementary bodies;
a dispensing unit for dispensing volumes of said test composition each comprising a predetermined maximum of elementary bodies, wherein the dispensing unit is a droplet generator for generating droplets of said test composition;
a MALDI TOF mass spectrometer apparatus configured for generating a mass spectrum of said test composition and comprising a flight path unit comprising:
a drying chamber, wherein solvent of the composition of the matrix material is evaporated to crystallize the matrix material onto solid matter, more particularly the elementary bodies, to generate an aerosol beam of coated elementary bodies, which drying chamber is arranged such that droplets dispensed from the droplet generator enter the drying chamber and dry during flight through the drying chamber;
an ionization chamber wherein said coated elementary bodies are irradiated with a laser to result in ionization of proteins and said ionized material is accelerated and passes a charged grid; and
a time-of-flight tube, wherein individual ions of the ionized material are separated;
wherein the MALDI TOF mass spectrometer apparatus furthermore comprises a detector for detecting the separated ions; and
a processor for generating a mean spectrum based on a plurality of mass spectrum of individual volumes and for comparing the mean spectrum with a reference, typically from a database.

42. The system as claimed in claim 41, further comprising optical image analysis means comprising an optical image recorder and processing means for analysis a recorded optical image.

43. The system as claimed in claim 42, further comprising droplet removal means, which are driven on the basis of said analysis by said processing means.

44. The system as claimed in claim 41, wherein the lysis means comprises sonication means.
